(19) United States
(12) Patent Application Publication
(10) Pub. No.: US 2022/0319004 A1
(43) Pub. Date: Oct. 6, 2022

(54) AUTOMATIC VESSEL ANALYSIS FROM 2D IMAGES

(71) Applicant: MEDHUB LTD, Tel Aviv (IL)

(72) Inventors: OR BRUCH-EL, TEL AVIV (IL); ALEXANDRE KASSEL, TEL AVIV (IL)

(21) Appl. No.: 17/836,112

(22) Filed: Jun. 9, 2022

Related U.S. Application Data
(63) Continuation-in-part of application No. PCT/IL2020/051276, filed on Dec. 10, 2020.
(60) Provisional application No. 62/945,896, filed on Dec. 10, 2019.

(30) Foreign Application Priority Data
Dec. 10, 2019 (IL) 271294

(51) Int. Cl. G06T 7/00; G06T 7/20; A61B 6/00; A61B 6/12; G16H 30/40

(52) U.S. Cl. CPC G06T 7/0012; G06T 7/20; A61B 6/504; A61B 6/507; A61B 6/5217; A61B 6/12; G16H 30/40; G06T 2207/10024; G06T 2207/10116; G06T 2207/30104; G06T 2207/20081; G06T 2207/30172

(57) ABSTRACT

A fully automated solution to vessel analysis based on image data including a system for analysis of a vessel that receives at least 2D images of a patient's vessels, the images obtained from two different angles during X-ray angiography, where the system uses color or grayscale features from a location of a stenosis in the images to provide an FFR value for the vessel.

### FIELD

The present invention relates to automated vessel analysis from 2D image data.

### BACKGROUND

Artery diseases involve circulatory problems in which narrowed arteries reduce blood flow to body organs. For example, coronary artery disease (CAD) is the most common cardiovascular disease, which involves reduction of blood flow to the heart muscle due to build-up of plaque in the arteries of the heart.

Current clinical practices used in the diagnosis of coronary artery disease include coronary angiography and/or non-invasive image-based methods such as computerized tomography (CT), which require constructing a 3D model of arteries from which a computer can create cross-sectional images (slices) of the imaged tissues, and which require an expert's interpretation of the images. Other image-based diagnostic methods typically require user input (e.g., a physician is required to mark vessels in an image) based on which further image analysis may be performed to detect pathologies such as lesions and stenoses.

### SUMMARY

Embodiments of the invention provide a fully automated solution to vessel analysis based on image data. A system, according to embodiments of the invention, detects a pathology and may provide a functional measurement value from a 2D image of a vessel, without having to construct a 3D model (i.e., without using CT techniques) and without requiring user input regarding the vessel and/or location of the pathology. Thus, embodiments of the invention enable detecting pathologies and providing a functional measurement value from 2D lengthwise images of a vessel obtained during X-ray angiography, as opposed to 2D cross section images that are used in CT procedures, such as CT angiography (CTA). Consequently, embodiments of the invention enable vessel analysis while exposing a patient to a significantly lower radiation dose compared with the level of radiation used during CT. Additionally, embodiments of the invention enable real-time analysis and treatment (e.g., stenting) of vessels, whereas analysis of CT generated images cannot be done in real-time and does not enable real-time treatment of vessels.

In one embodiment, a system for analysis of a vessel includes a processor to receive at least two 2D images of a stenosis in a patient's vessel, the images typically being 2D lengthwise images obtained during X-ray angiography, each of the images captured from a different angle. The processor determines the location of the stenosis in the vessel in each of the images and calculates an FFR value of the vessel or for the stenosis, based on a color or grayscale feature, extracted from the location of the stenosis from each of the images. Features extracted from the “location of the stenosis” may be features extracted from pixels of the stenosis itself and/or surrounding pixels and/or pixels in the vicinity of the stenosis.

The processor, which may be in communication with a user interface device, may output to a user (e.g., display on the user interface device) an indication of the FFR value.

The processor may input the color or grayscale feature into a machine learning model to predict the FFR value. In addition to the color or grayscale feature, the processor may input into the machine learning model additional features, such as, a shape feature and/or a morphological feature, to calculate the FFR value based on the color or grayscale feature and on one or more of these additional features.

In embodiments of the invention, the location of the stenosis in the vessel is a location of the stenosis relative to a structure of the vessel. The processor may apply a classifier on a first image to determine the location of the stenosis and then the processor may determine the location of the stenosis in the vessel in a second image by tracking the stenosis to the second image, based on the determined location of the stenosis relative to the structure of the vessel.

The processor may attach a virtual mark to the stenosis to track the stenosis from the first image to the second of image based on the virtual mark. The mark may be based on the location of the stenosis relative to the structure of the vessel.

Embodiments of the invention determine, in each image, a location of a pathology (e.g., stenosis) relative to a structure of the vessel. This enables tracking the same pathology throughout different angiogram images, even if the images were captured from different angles (e.g., due to rotation of the X-ray imaging device and/or rotation of the patient which causes the pathology to appear different in every image). Thus, embodiments of the invention enable automatic analysis of a vessel, such as, determination of functional measurements (e.g., FFR values) from images obtained during X-ray angiography, with no need for user (e.g., physician) input.

### BRIEF DESCRIPTION OF THE FIGURES

The invention will now be described in relation to certain examples and embodiments with reference to the following illustrative figures so that it may be more fully understood. In the drawings:

FIG. 1 schematically illustrates a system for analysis of a vessel, according to embodiments of the invention;

FIG. 2 schematically illustrates a method for automatically indicating a location of a stenosis on an image of a patient's vessels, according to an embodiment of the invention;

FIGS. 3A and 3B schematically illustrate images of vessels analyzed according to embodiments of the invention;

FIG. 4 schematically illustrates a method for tracking a pathology throughout images of vessels, according to an embodiment of the invention; and

FIG. 5 schematically illustrates a method for providing a functional measurement for a pathology, according to embodiments of the invention.

### DETAILED DESCRIPTION

Embodiments of the invention provide methods and systems for automated analysis of vessels from images of the vessels, or portions of the vessels, and display of the analysis results.

Analysis, according to embodiments of the invention, may include diagnostic information, such as presence of a pathology, identification of the pathology, location of the pathology, etc. Analysis may also include functional measurements, such as estimates of FFR values. The analysis results, may be displayed to a user.

A “vessel” may include a tube or canal in which body fluid is contained and conveyed or circulated. Thus, the term vessel may include blood veins or arteries, coronary blood vessels, lymphatics, portions of the gastrointestinal tract, etc.

An image of a vessel may be obtained using suitable imaging techniques, for example, X-ray imaging, ultrasound imaging, Magnetic Resonance imaging (MRI) and others suitable imaging techniques. Embodiments of the invention use angiography, which includes injecting a radio-opaque contrast agent into a patient's blood vessel and imaging the blood vessel using X-ray based techniques. The images obtained, according to embodiments of the invention, are typically 2D lengthwise images of a vessel, as opposed to, for example, 2D cross section images that are used in methods that require constructing a 3D model of the vessel, such as CTA and other CT methods.

A pathology may include, for example, a narrowing of the vessel (e.g., stenosis or stricture), lesions within the vessel, etc.

A “functional measurement” is a measurement of the effect of a pathology on flow through the vessel. Functional measurements may include measurements such as an estimate of fractional flow reserve (FFR), an estimate of instant flow reserve (iFR), coronary flow reserve (CFR), quantitative flow ratio (QFR), resting full-cycle ratio (RFR), quantitative coronary analysis (QCA), and more.

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Unless specifically stated otherwise, as apparent from the following discussions, it is appreciated that throughout the specification discussions utilizing terms such as “using”, “analyzing”, “processing,” “computing,” “calculating,” “determining,” “detecting”, “identifying” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices. Unless otherwise stated, these terms refer to automatic action of a processor, independent of and without any actions of a human operator.

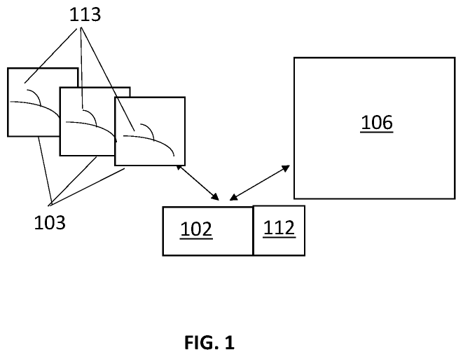

In one embodiment, which is schematically illustrated in FIG. 1, a system for analysis of a vessel includes a processor 102 in communication with a user interface device 106. Processor 102 receives one or more images 103 of a vessel 113. The images 103 may be consecutive images, typically forming a video that can be displayed via the user interface device 106. At least some of the images 103 may be capturing the vessel 113 from different angles.

Processor 102 performs analysis on the received image(s) and communicates analysis results and/or instructions or other communications, based on the analysis results, to a user via the user interface device 106. In some embodiments, user input can be received at processor 102, via user interface device 106.

Vessels 113 may include one or more vessel or portion of a vessel, such as a vein or artery, a branching system of arteries (arterial trees) or other portions and configurations of vessels.

Processor 102 may include, for example, one or more processors and may be a central processing unit (CPU), a graphics processing unit (GPU), a digital signal processor (DSP), a field-programmable gate array (FPGA), a microprocessor, a controller, a chip, a microchip, an integrated circuit (IC), or any other suitable multi-purpose or specific processor or controller. Processor 102 may be locally embedded or remote, e.g., on the cloud.

Processor 102 is typically in communication with a memory unit 112. In one embodiment the memory unit 112 stores executable instructions that, when executed by the processor 102, facilitate performance of operations of the processor 102, as described below. Memory unit 112 may also store image data (which may include data such as pixel values that represent the intensity of light having passed through body tissue and/or light reflected from tissue or from a contrast agent within vessels, and received at an imaging sensor, as well partial or full images or videos) of at least part of the images 103.

Memory unit 112 may include, for example, a random access memory (RAM), a dynamic RAM (DRAM), a flash memory, a volatile memory, a non-volatile memory, a cache memory, a buffer, a short term memory unit, a long term memory unit, or other suitable memory units or storage units.

The user interface device 106 may include a display, such as a monitor or screen, for displaying images, instructions and/or notifications to a user (e.g., via graphics, images, text or other content displayed on the monitor). User interface device 106 may also be designed to receive input from a user. For example, user interface device 106 may include or may be in communication with a mechanism for inputting data, such as a keyboard and/or mouse and/or touch screen, to enable a user to input data.

All or some of the components of system may be in wired or wireless communication, and may include suitable ports such as USB connectors and/or network hubs.

In one embodiment, processor 102 detects a location of a pathology, such as a stenosis, within a 2D image of a patient's vessels. Thus, processor 102 may automatically indicate the actual location of a stenosis on an image of a patient's vessels, e.g., on an X-ray image.

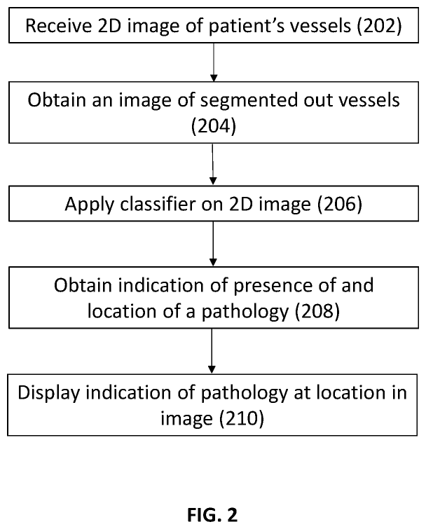

In one example, which is schematically illustrated in FIG. 2, processor 102 receives a 2D image of a patient's vessels (e.g., an angiogram image) (step 202) and applies on the image algorithms for segmenting the image (e.g., semantic segmentation algorithms and/or machine learning models, as described below), to obtain an image of segmented out vessels, also referred to as a vessels mask (step 204). Processor 102 then applies a classifier on the image of the segmented out vessels (step 206) to obtain, from output of the classifier (assisted by using the vessels mask), an indication of a presence of a pathology (e.g., stenosis) in the vessels and a location of the pathology (step 208). The location may be an x,y location on a coordinate system describing the image and/or the location may be a description of the section of the vessel where the pathology is located.

Classifiers, such as DenseNet, CNN (Convolutional Neural Network) or EfficientNet, may be used to obtain a determination of presence of a pathology and to determine the location of the pathology. Classifiers, according to embodiments of the invention, may be pre-trained on training data that includes 2D (typically lengthwise) X-ray angiogram images of vessels which may include a pathology (e.g., stenosis). In one embodiment the training data includes X-ray angiography images that include a stenosis and, optionally, X-ray angiography images that do not include a stenosis. The neural network composing the classifier may be learned by a scheme of supervised learning, or possibly semi-supervised learning. In the training of the neural network, training data is repeatedly input to the neural network and an error of an output of the neural network for the training data and a target, is calculated, and the error of the neural network is back-propagated in order to decrease the error and update the neural network. In the case of supervised learning, training data, which includes 2D X-ray angiogram images (e.g., images of a single vessel (with and possibly without stenoses) obtained from different points of view and/or images of different vessels with and possibly without stenoses), is labelled with a correct answer (e.g., stenosis exists/does not exist in the image).

Applying a classifier on an angiography image enables detection of a pathology by using computer vision techniques, without requiring user input regarding a location of the vessels in the image and/or location of the pathology.

Processor 102 may then cause an indication of the pathology to be displayed, via the user interface device 106, on the image of the patient's vessels (e.g., image 103), at the location of the pathology (step 210). In some embodiments the indication of pathology can be displayed at a same location on a plurality of consecutive images (e.g., a video angiogram).

An indication of a pathology displayed on a display of a user interface device may include, for example, graphics, such as, letters, numerals, symbols, different colors and shapes, etc., that can be superimposed on the image or video of the patient's vessels.

In some embodiments, processor 102 determines a probability of presence of the pathology, e.g., based on output of the classifier, and causes an indication of the pathology to be displayed only if the probability is above a predetermined threshold.

In some embodiments, processor 102 obtains a vessels mask by using semantic segmentation algorithms on the image. A machine learning model can be used for the segmentation, e.g. deep learning models such as Unet or FastFCN or other deep learning based semantic segmentation techniques.

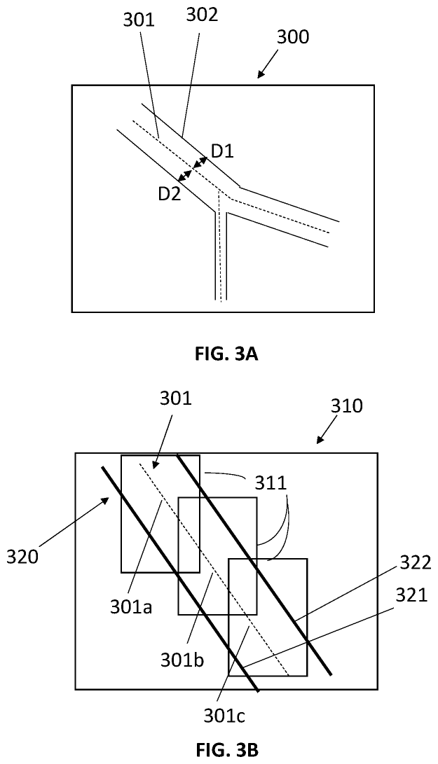

FIG. 3A. schematically illustrates a vessels mask image 300 including vessels 302. Processor 102 may determine a centerline 301 of the vessels 302 and may input to the classifier described above, a distance between the centerline 301 and a border of the vessels, e.g. distance D1 and/or D2. The classifier may be applied on a plurality of portions of image 300, each portion including a different part of the centerline 301. The classifier may use the input distances D1 and/or D2 to determine presence of a pathology in the vessels and a location of the pathology.

In other embodiments, one example of which is schematically illustrated in FIG. 3B, the classifier is applied on a plurality of portions 311 of a lengthwise image 310 of a vessel 320, without input of distances D1 or D2 or input of any other measurement. In these embodiments, the classifier, optionally based on or including a deep neural network, accepts as input only an image (e.g., image 310), or portions of an image (e.g., portions 311) of a vessel and outputs an analysis of the vessel (e.g., the presence and location of a pathology in the vessel) based on the input image(s).

In some embodiments, the classifier may be applied on a plurality of partially overlapping portions of an image of a vessel (possibly, a vessel mask image) and may output an analysis of the vessel based on the partially overlapping portions of image. In one embodiment, the plurality of portions 311 of image 310, on which the classifier is applied, each include a different part of the centerline 301, such as parts 301a, 301b and 301c, where each of the different parts of the centerline may partially overlap another part of the centerline. For example, part 301a partially overlaps part 301b and part 301b partially overlaps parts 301a and 301c. This ensures that each portion of image input to the classifier includes a full portion of the vessel (typically including both borders 321 and 322) such that a possible stenosis will be located more or less in the center parts of the portion of image and not at the periphery of the portion of image, where it may be cut off or otherwise not clearly presented.

Determining a centerline as well as calculating distances D1 and D2 can be done by using known algorithms for medial axis skeletonization, for example, scikit-image algorithms.

In some embodiments the 2D image (from which a vessels mask can be obtained) is an optimal image, selected from a plurality of 2D images of the patient's vessels, as the image showing the most detail. In the case of angiogram images, which include contrast agent injected to a patient to make vessels (e.g., blood vessels) visible on an X-ray image, an optimal image may be an image of a blood vessel showing a large/maximum amount of contrast agent. Thus, an optimal image can be detected by applying image analysis algorithms (e.g., to detect the image frames having the most colored pixels) on a sequence of images.

In one embodiment, an image captured at a time corresponding with maximum heart relaxation is an image showing a maximum amount of contrast agent. Thus, an optimal image may be detected based on capture time of the images compared with, for example, measurements of electrical activity of the heartbeat (e.g., ECG printout) of the patient.

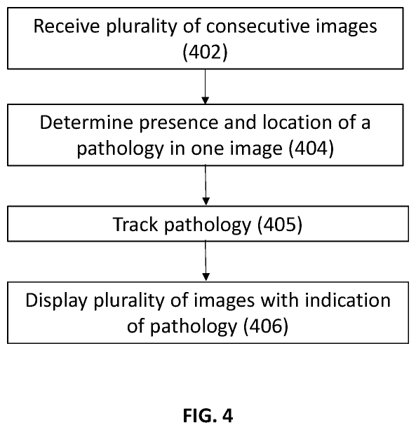

In one embodiment, which is schematically illustrated in FIG. 4, processor 102 receives a plurality of consecutive images of a patient's vessels (step 402). Processor 102 determines presence and location of a pathology (such as a stenosis) in the vessels in one image from the plurality of images (step 404), e.g., by applying a machine learning model on one of the images and applying a classifier on the images, as described above. Processor 102 then causes an indication of pathology to be displayed, via the user interface device 106, at the determined location on a plurality (possibly, on each) of the consecutive images (step 406).

In some embodiments, once a pathology is detected in a first image from the plurality of images, the pathology may be tracked throughout the plurality of images (e.g., video) (step 405), such that the same pathology can be detected in each of the images, even if it's shape or other visual characteristics change in between images.

One method of tracking a pathology may include attaching a virtual mark to the pathology detected in the first image. In some embodiments the virtual mark is location based, e.g., based on location of the pathology within portions of the vessel. In some embodiments, a virtual mark includes the location of the pathology relative to a structure of the vessel. A structure of a vessel can include any visible indication of anatomy of the vessel, such as junctions of vessels and/or specific vessels typically present in patients. Processor 102 may detect the vessel structure in the image by using computer vision techniques (such as by using the vessel mask described above), and may then index a detected pathology based on its location relative to the detected vessel structures.

For example, a segmenting algorithm can be used to determine which pixels in the image are part of the pathology and the location of the pathology relative to structures of the vessel can be recorded, e.g., in a lookup table or other type of virtual index. For example, in a first image a stenosis is detected at a specific location (e.g., in the distal left anterior descending artery (LAD)). A stenosis located at the same specific location (distal LAD) in a second image, is determined to be the same stenosis that was detected in the first image. If, for example, more than one stenosis is detected within the distal LAD, each of the stenoses are marked with their relative location to additional structures of the vessel, such as, in relation to a junction of vessels, enabling to distinguish between the stenoses in a second image.

Thus, the processor 102 creates a virtual mark which is specific per pathology, and in a case of multiple pathologies in a single image, distinguishes the multiple pathologies from one another. The pathology can then be detected in following images of the vessel, based on the virtual mark.

In some embodiments, processor 102 may assign a name or description to a pathology based on the location of the pathology within the vessel and the indication of pathology can include the name or description assigned to the pathology.

In one embodiment, the processor can calculate a value of a functional measurement, such as an FFR estimated value, for each pathology and may cause the value(s) to be displayed.

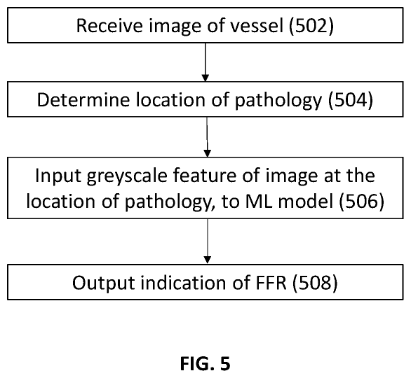

As schematically illustrated in FIG. 5, processor 102 receives an image of a patient's vessels (step 502) and determines a location of a pathology in the vessels based on the image (step 504), e.g., as described above. Processor 102 then calculates a functional measurement of the vessel based on a color feature (which may include color or grayscale) of the image at the location of the stenosis, e.g., by inputting the color feature into a machine learning model that predicts a value of the functional measurement (step 506). Color features may be extracted from a location of the stenosis, which may include pixels of the stenosis itself and/or surrounding pixels and/or pixels in the vicinity of the stenosis.

In one embodiment, a machine learning model running a regression algorithm is used to predict a value of a functional measurement (e.g., FFR estimate) from an image of the vessel, namely, based on a color feature of the image at the location of a pathology. For example, the machine learning algorithm can be implemented by using the XGBoost algorithm or other Gradient Boosted Machine or decision trees regression. In other examples, neural network or deep neural network based regression can be used.

Processor 102 then outputs an indication of the functional measurement to a user (step 508), e.g., via user interface device 106.

In some embodiments, at least two images (typically, 2D images obtained during X-ray angiography, each image captured from a different angle) of the same stenosis in a patient's vessel, are used. Processor 102 determines a location of the stenosis in the vessel in each of the images and calculates an FFR value of the vessel/stenosis based on a color or grayscale feature extracted from each of the images, at the location of the stenosis.

The image of the patient's vessels may be a grayscale image and the color feature may include shades of grey. Other features may be input to the machine learning model in addition to the color features, for example, morphological features (e.g., branching system of arteries (arterial trees) or other portions and configurations of vessels) and/or shape features.

Thus, processor 102 determines a functional measurement directly from an image, e.g., by employing machine learning models and classifiers as described above, with no need for user input.

Since, as described herein, the same stenosis may be tracked throughout different images (even images captured from different points of view or angles), color or grayscale features can be easily extracted from the location of the same stenosis in each of the different images. Thus, an FFR value (or other functional measurement) for a specific stenosis may be provided based on color features from two or more images of the specific stenosis.

In some embodiments, FFR estimate and/or other functional measurements can be obtained during or after stenting, by using the systems and methods described above, namely, obtaining an image of the patient's vessels during or after stenting and calculating a functional measurement of the vessel based on a color feature of the image at the location of the stent (e.g., at the stents ends and/or within the stent). Thus, in one embodiment, a method for analysis of a vessel during or after stenting, may include receiving an angiogram image of a patient's vessel with a stent, automatically determining a location of the stent in the vessel, e.g., based on image analysis, as described herein, and calculating an FFR value of the vessel (e.g., at the location of the stent) based on a color or grayscale feature of the image, at the location of the stent. An indication of the FFR value may then be output to the user.

Obtaining functional measurements during or after stenting provides information in real-time regarding the success of the stenting procedure.

What is claimed is:

1. A system for analysis of a vessel, the system comprising a processor configured to:
receive at least two 2D images of a stenosis in a patient's vessel, the images obtained during X-ray angiography, each image captured from a different angle;
determine a location of the stenosis in the vessel in each of the images;
calculate an FFR value of the vessel based on a color or grayscale feature of each of the images, at the location of the stenosis; and
output to a user an indication of the FFR value.

2. The system of claim 1 wherein the processor inputs the color or grayscale feature into a machine learning model, the model to predict the FFR value.

3. The system of claim 2 wherein the processor inputs a shape feature into the machine learning model to calculate the FFR value based on the color or grayscale feature and on the shape feature.

4. The method of claim 2 wherein the processor inputs a morphological feature into the machine learning model to calculate the FFR value based on the color or grayscale feature and on the morphological feature.

5. The system of claim 1 wherein the location of the stenosis in the vessel comprises a location of the stenosis relative to a structure of the vessel and wherein the processor is to apply a classifier on a first image from the at least two images to determine the location of the stenosis.

6. The system of claim 5 wherein the processor determines the location of the stenosis in the vessel in each of the images by determining the location of the stenosis in the vessel in a first image; and tracking the stenosis to a second image, based on the determined location of the stenosis relative to the structure of the vessel.

7. The system of claim 6 wherein the processor attaches a virtual mark to the stenosis to track the stenosis from the first image to the second of image based on the virtual mark.

8. The system of claim 7 wherein the virtual mark is based on the location of the stenosis relative to the structure of the vessel.

9. The system of claim 5 wherein the processor is to assign to the stenosis a description including a name of the vessel and section of the vessel in which the stenosis is located.

10. The system of claim 5 wherein the processor applies on the images an algorithm for segmenting, to obtain an image of segmented out vessels and applies the classifier on the images of segmented out vessels.

11. The system of claim 5 wherein the classifier is applied on a plurality of different portions of each of the images.

12. The system of claim 5 wherein the processor determines a centerline of the vessel in the images and wherein the classifier is applied on a plurality of image portions, each portion including a different part of the centerline.

13. The system of claim 12 wherein the processor inputs a distance between the centerline and a boarder of the vessel, to the classifier, to determine the location of the stenosis.

14. The system of claim 5 wherein the first image is selected from a plurality of angiography images of the patient's vessels, as an image showing the most detail.

15. The system of claim 1 wherein the processor causes the FFR value to be displayed to a user.

16. A method for analysis of a vessel during or after stenting, the method comprising:
receiving an angiogram image of a patient's vessel with a stent;
automatically determining a location of the stent in the vessel; and
calculating an FFR value of the vessel based on a color or grayscale feature of the image, at the location of the stent; and
outputting to a user an indication of the FFR value.

17. The method of claim 16 wherein the location of the stent comprises one or both of: an end of the stent, a location within the stent.
